(19) United States
(12) Patent Application Publication
(10) Pub. No.: US 2018/0028633 A1
(43) Pub. Date: Feb. 1, 2018

(54) CHIMERIC ANTIGEN RECEPTOR COMBINATION THERAPY FOR TREATING TUMORS

(71) Applicant: Lan Bo Chen, Lexington, MA (US)

(72) Inventor: Lan Bo Chen, Lexington, MA (US)

(21) Appl. No.: 15/664,151

(22) Filed: Jul. 31, 2017

Related U.S. Application Data
(60) Provisional application No. 62/368,655, filed on Jul. 29, 2016.

(51) Int. Cl. A61K 39/00; A61K 47/68; C07K 14/725; C07K 16/18

(52) U.S. Cl. CPC A61K 39/0011; C07K 16/18; A61K 47/6813; C07K 14/7051; C07K 2317/622; C07K 2317/64; C07K 2317/31; C07K 2317/24; C07K 2319/74; A61K 2039/5156

(57) ABSTRACT

A method for treating a tumor in a subject by administering at least three of the following treatment modalities: (i) an antibody, (ii) T cells bearing a first chimeric antigen receptor (CAR), (iii) NK cells bearing a second CAR, and (iv) NKT cells bearing a third CAR. The antibody binds specifically to stage-specific embryonic antigen 4 and each CAR contains a scFv that also binds specifically to SSEA4.

### CROSS-REFERENCE TO RELATED APPLICATIONS

The present application claims priority to Provisional Application No. 62/368,655, filed on Jul. 29, 2016. The content of this prior application is hereby incorporated by reference in its entirety.

### BACKGROUND

Targeted cancer immunotherapy, as compared to chemotherapy, holds the promise of better efficacy, both short-term and long-term, with fewer side effects.

For example, therapeutic antibodies have been developed that specifically bind to carbohydrate antigens on tumor cells, resulting in death of the cells via recruitment and stimulation of T cells. These carbohydrate antigens, e.g., stage-specific embryonic and stage-specific embryonic antigen 4 (“SSEA4”), are expressed in a wide variety of tumor types and are not expressed in most adult tissues. See, e.g., Lee et al. 2014, J. Am. Chem. Soc. 136:16844-16853. The effectiveness of therapeutic antibodies is often limited due to suppression of T cell activity by tumor cells.

Recently, chimeric antigen receptors (“CARs”) have been developed to program T cells, NK cells, and NKT cells to attack tumor cells bearing a particular tumor antigen. A CAR contains an extracellular domain that binds to the tumor antigen and one or more intracellular domains that provide both primary and co-stimulatory signals to the T cells, NK cells, and NKT cells. These cells can be engineered in vitro to express CAR having an extracellular domain of choice.

The CAR approach has proven to be effective, yet not without serious side effects. In an example, infusion of large numbers of T cells expressing CAR causes graft-versus-host disease, in which the T cells attack non-malignant tissues.

There is a need to develop combinatorial CAR-based tumor therapies that are safer and more effective than those currently in use.

### SUMMARY

To meet this need, a method for treating a tumor in a subject is disclosed in which a subject having a tumor is administered at least three of the following treatment modalities: (i) an antibody, (ii) T cells bearing a first chimeric antigen receptor (CAR), (iii) NK cells bearing a second CAR, and (iv) NKT cells bearing a third CAR. The antibody binds specifically to stage-specific embryonic antigen 4 (SSEA4); the T cells, NK cells, and NKT cells are autologous cells; the first, second, and third CARs each contain a scFv that binds specifically to SSEA4; and the tumor expresses SSEA4.

The details of one or more embodiments of the invention are set forth in the description below. Other features, objects, and advantages of the invention will be apparent from the description and from the claims.

Importantly, all documents cited herein are hereby incorporated by reference in their entirety.

### DETAILED DESCRIPTION

As mentioned above, the method of the invention includes administering at least three of the following four treatment modalities, i.e., an antibody, T cells bearing a first CAR, NK cells bearing a second CAR, and NKT cells bearing a third CAR. These treatment modalities can be administered together or each individually. For example, the antibody can be administered first, followed by the T cells 1, 2, 3, or 4 weeks later. In another example, the antibody is administered first, followed by T cells and NK cells together. In an additional example, an antibody is administered first, T cells are administered second, and NK cells are administered third. These administrations can be separated temporally.

As mentioned above, the method of the invention, in one embodiment, includes administering to a subject an antibody that specifically binds to SSEA4.

Examples of an antibody that specifically binds to SSEA4 include a chimeric anti-SSEA4 antibody and a fully humanized anti-SSEA4 monoclonal antibody. See US Patent Application Publication 2016/0102151 for examples of anti-SSEA4 antibodies for use in the method of the invention.

The anti-SSEA4 antibody can be linked to a cytokine, a cytotoxic agent, a modified immunoglobulin Fc domain, anti-CD3, or anti-CD16.

A cytokine can be fused to the anti-SSEA4 antibody as part of a fusion protein. See Kiefer et al. 2016, Immunol. Revs. 270:178-192. In another example, the cytokine is linked to the anti-SSEA4 antibody via cross-links between lysine residues. Exemplary suitable cytokines include G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

Exemplary cytotoxic agents are diphtheria toxin, pseudomonas exotoxin A (“PE38”), doxorubicin, methotrexate, an auristatin, a maytansine, a calicheamicin, a duocarmycin, a pyrrolobenzodiazepine dimer, and 7-ethyl-10-hydroxy-camptothecin. Suitable cytotoxic agents are described in Peters et al. 2015, Biosci. Rep. 35:1-20 (“Peters et al”); Bouchard et al. 2014, Bioorg. Med. Chem. Lett. 24:5357-5363; Panowski et al. 2014, mAbs 6:34-45; and Mazor et al. 2016, Immunol. Revs. 270:152-164.

The cytotoxic agent can be linked to the anti-SSEA4 antibody via a linker. In an embodiment, the linker is cleavable such that, upon internalization of the bifunctional agent by a tumor cell, the cytotoxic agent is cleaved from the binding domain. Examples of a cleavable linker include, but are not limited to, acid-labile small organic molecules (e.g., hydrazone), protease cleavable peptides (e.g., valine-citrulline dipeptide), and disulfide bonds. In another embodiment, the linker is not cleavable. In this case, the cytotoxic agent is released upon degradation of the anti-SSEA4 antibody linked to it. Additional examples of linkers are described in Peters et al.

If the cytotoxic agent is a protein, it can be linked to the anti-SSEA4 antibody or antibody fragment via a peptide bond, e.g., as part of a fusion protein. In a particular example, PE38 can be fused to the C-terminus of a V_{L} chain of an anti-SSEA4 monoclonal antibody.

In a particular embodiment, the anti-SSEA4 antibody is an anti-SSEA4 antibody fragment linked to a modified immunoglobulin Fc domain. For example, the Fc domain can be modified such that it specifically targets the FcγRIIa receptor, the FcγRIIIa receptor, or the FcRn receptor, as compared to an unmodified Fc domain. Targeting the FcγRIIa or FcγRIIIa receptor leads to an increased cytotoxic immune response. On the other hand, targeting the FcRn receptor increases the half-life of the anti-SSEA4 antibody. Modifications to the Fc domain that increase its affinity for the FcγRIIa receptor, the FcγRIIIa receptor, or the FcRn receptor are described in Moore et al. 2010, mAbs 2:181-189 and Lobner et al. 2016, Immunol. Revs. 270:113-131.

In another embodiment, the anti-SSEA4 antibody is linked to an anti-CD3 molecule. The anti-CD3 molecule activates T cells localized to tumor cells via the anti-SSEA4 antibody. An exemplary anti-CD3 molecule is an antibody fragment. The anti-CD3 molecule can specifically bind to CD3ε. Further, a scFv that specifically binds to SSEA4 can be fused to another scFv that specifically binds to CD3.

In still another embodiment, the anti-SSEA4 antibody is linked to an anti-CD16 molecule. The anti-CD16 molecule activates NK cells localized to tumor cells via the anti-SSEA4 antibody. Like the anti-CD3 molecule described in the preceding paragraph, the anti-CD16 molecule can be an antibody fragment that binds specifically to CD16. Exemplary constructs are an anti-SSEA4/anti-CD16 chimeric antibody and a scFv that specifically binds to SSEA4 fused to another scFv that specifically binds to CD16.

As set forth, supra, the subject can be administered with up to three types of cells, i.e., T cells, NK cells, and NKT cells, bearing a CAR containing a scFv that binds specifically to SSEA4. Each of the T cells, NK cells, and NKT cells can express on their surfaces the same CAR. Alternatively, the T cells can express a different CAR than the NK cells or NKT cells. In one embodiment, each of these three cell types express a different CAR.

The scFv that specifically binds to SSEA4 can be, e.g., any of those exemplified in US Patent Application Publication 2016/0102151.

In addition to the scFv, the CAR also contains an endodomain from CD3ζ or FcεRIγ. The endodomain contains one or more immunoreceptor tyrosine-based activating motifs (“ITAM”).

The CAR further includes a hinge/spacer region and a transmembrane region between the scFv and the endodomain.

Exemplary sequences that can be used as a hinge/spacer region are derived from the hinge region of, e.g., IgG1, IgG4, and IgD. Alternatively, it can be derived from CD8. See, e.g., Dai et al. 2016, J. Natl. Cancer Inst. 108:1-14 (“Dai et al.”) and Shirasu et al., 2012, Anticancer Res. 32:2377-2384 (“Shirasu et al.”).

Exemplary transmembrane regions that can be included in the CAR are derived from CD3ζ, CD4, CD8, or CD28. See Dai et al. and Shirasu et al.

Optionally, the CAR also contains a second endodomain in addition to the endodomain from CD3ζ or FcεRIγ. The second endodomain, e.g., from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, and PILR, like the first endodomain, contains one or more ITAM.

Furthermore, the CAR can contain a third endodomain, which also can be from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR. The third endodomain is different from the second endodomain.

In a specific embodiment, the CAR contains an anti-SSEA4 scFv fused to a spacer/hinge from CD8 that is fused to a transmembrane domain also from CD8 fused to the N-terminus of the endodomain from CD28, which in turn is fused to the N-terminus of the endodomain from CD137, which in turn is fused to the N-terminus of the endodomain from CD3ζ.

In one embodiment, the method of the invention can include a step of obtaining T cells, NK cells, or NKT cells bearing any of the CAR described above. This can be accomplished by transducing T cells, NK cells, or NKT cells in vitro with an expression vector encoding the CAR.

The expression vector includes a promoter operably linked to a nucleic acid encoding the CAR. The promoter is active in T cells, NK cells, or NKT cells.

Exemplary CAR expression vectors based on lentiviral vectors or a gamma retroviral vectors are set forth in Dai et al.; Jin et al. 2016, EMBO Mol. Med. 8:702-711; Liechtenstein et al. 2013, Cancers 5:815-837; and Schonfeld et al. 2015, Mol. Therapy 23:330-338 (“Schonfeld”).

Such expression vectors are used for integrating the promoter/CAR-encoding nucleic acid into T cell, NK cell or NKT cell genomic DNA to produce stable expression of the CAR.

Alternatively, the expression vector contains sequences that facilitate transposon-mediated genomic integration of the promoter/CAR-encoding nucleic acid into T cells, NK cells, or NKT cells. Examples of these expression vectors are the so-called “PiggyBac” and “Sleeping Beauty” expression vectors. See Nakazawa et al. 2011, Mol. Ther. 19:2133-2143 and Sourindra et al. 2013, J. Immunotherapy 36:112-123.

T cells, NK cells, or NKT cells are isolated from a subject suffering from a tumor. Procedures for isolating these cells are known in the art. See, e.g., Kaiser et al. 2015, Cancer Gene Therapy 22:72-78 (“Kaiser et al.”).

Established NK cell lines can also be used in the method instead of NK cells isolated from a subject. See, e.g., Schonfeld.

Expression vectors are transduced into T cells, NK cells, or NKT cells by, e.g., electroporation, lipofection, lentiviral infection, and gamma retrovirus infection.

The transduced cells are expanded in vitro, using methods known in the art. See Kaiser et al.

Finally, the expanded T cells, NK cells, or NKT cells are administered by infusion in one batch or in two or more batches into the subject having a tumor.

In one embodiment, the method of the invention includes a preconditioning step that is performed prior to the just-mentioned administering step. The preconditioning step is accomplished by treating the subject with a drug that induces lymphodepletion. Examples of these drugs include cyclophosphamide and fludarabine. Additional drug examples can be found in Dai et al. and Han et al. 2013, J. Hematol. Oncol. 6:47-53.

The method above for treating a tumor is effective for treating e.g., a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

Without further elaboration, it is believed that one skilled in the art can, based on the description above, utilize the present invention to its fullest extent.

The following references, some cited supra, can be used to better understand the background of the application:
Abate-Daga et al., Mol. Ther. Oncolytics 3:1-7.
Bouchard et al. 2014, Bioorg. Med. Chem. Lett. 24:5357-5363
Becker et al. 2010, J. Immunol. 184:6822-6832
Curran et al. 2012, J. Gene Med. 14:405-415
Dai et al. 2016, J. Natl. Cancer Inst. 108:1-14
Guest et al., 2005, J. Immunother. 28:203-211
Han et al. 2013, J. Hematol. Oncol. 6:47-53
Heczey et al. 2014, Blood 124:2824-2833
James et al. 2008, J. Immunol. 180:7028-7038.
Kaiser et al. 2015, Cancer Gene Therapy 22:72-78.
Kiefer et al. 2016, Immunol. Revs. 270:178-192
Lawson 2012, Immunology 137:20-27
Lee et al. 2014, J. Am. Canc. Soc. 136:16844-16853.
Lobner et al. 2016, Immunol. Revs. 270:113-131
Mazor et al. 2016, Immunol. Revs. 270:152-164
Moore et al. 2010, mAbs 2:181-189
Moritz et al. 1995 Gene Therapy 2:539-546
Nakazawa et al. 2011, Mol. Ther. 19:2133-2143
Panowski et al. 2014, mAbs 6:34-45
Pegram et al. 2011, Immunol. Cell Biol. 89:216-224
Peters et al. 2015, Biosci. Rep. 35:1-20
Rajagopalan et al. 2005, J. Exp. Med. 201:1025-1029
Rezvani et al. 2015, Front. Immunol. 17 November
Rodgers et al. 2016, Proc. Natl. Acad. Sci. January 12:E459-E468
Ruggeri et al. 2002, Science 295:2097-2100
Schonfeld et al. 2015, Mol. Therapy 23:330-338
Shirasu et al. 2012, Anticancer Res. 32:2377-2384
Sourindra et al., 2013, J. Immunotherapy 36:112-123

The contents of the above references are hereby incorporated by reference in their entirety.

### Other Embodiments

All of the features disclosed in this specification may be combined in any combination. Each feature disclosed in this specification may be replaced by an alternative feature serving the same, equivalent, or similar purpose. Unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating a tumor in a subject, the method comprising administering to a subject having a tumor at least three treatment modalities selected from the group consisting of an antibody, T cells bearing a first chimeric antigen receptor (CAR), NK cells bearing a second CAR, and NKT cells bearing a third CAR, wherein the antibody binds specifically to stage-specific embryonic antigen 4 (SSEA4); the T cells, NK cells, and NKT cells are autologous cells; the first, second, and third CARs each contain a scFv that binds specifically to SSEA4; and the tumor expresses SSEA4.

2. The method of claim 1, wherein each of the first, second, and third CARs contains, independently, a first endodomain from CD3ζ or FcεRIγ.

3. The method of claim 2, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

4. The method of claim 3, wherein the antibody is linked to an agent selected from the group consisting of a cytokine, a cytotoxic agent, a modified immunoglobulin Fc domain, anti-CD3, and anti-CD16.

5. The method of claim 4, wherein the antibody is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

6. The method of claim 4, wherein the antibody is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

7. The method of claim 4, wherein the antibody is linked to a modified immunoglobulin Fc domain modified to target the FcγRIIa receptor, the FcγRIIIa receptor, or the FcRn receptor.

8. The method of claim 4, wherein the antibody is linked to anti-CD3 or anti-CD16.

9. The method of claim 2, wherein each of the first, second, and third CAR further contains, independently, a second endodomain from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR.

10. The method of claim 9, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

11. The method of claim 10, wherein the antibody is linked to an agent selected from the group consisting of a cytokine, a cytotoxic agent, a modified immunoglobulin Fc domain, anti-CD3, and anti-CD16.

12. The method of claim 11, wherein the antibody is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

13. The method of claim 11, wherein the antibody is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

14. The method of claim 11, wherein the antibody is linked to a modified immunoglobulin Fc domain modified to target the FcγRIIa receptor, the FcγRIIIa receptor, or the FcRn receptor.

15. The method of claim 11, wherein the antibody is linked to anti-CD3 or anti-CD16.

16. The method of claim 9, wherein each of the first, second, and third CAR further contains, independently, a third endodomain from CD28, CD137, CD4, OX40, ICOS, Ly49D, Ly49H, KIR2DL4, KIR2DS1, KIR2DS2, KIR2DS3, KIR2DS4, KIR2DS5, KIR3DS1, NKG2C, NKG2E, NKG2D, NKp30, NKp44, NKp46, NKp80, DNAM-1, or PILR.

17. The method of claim 16, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.

18. The method of claim 17, wherein the antibody is linked to an agent selected from the group consisting of a cytokine, a cytotoxic agent, a modified immunoglobulin Fc domain, anti-CD3, and anti-CD16.

19. The method of claim 18, wherein the antibody is linked to a cytokine selected from the group consisting of G-CSF, GM-CSF, IFNγ, IFNα, IL-1β, IL-2, IL-4, IL-6, IL-7, IL-9, IL-12, IL-13, IL-15, IL-17, IL-21, IL-23, and TNF.

20. The method of claim 18, wherein the antibody is linked to a cytotoxic agent selected from the group consisting of Diphtheria toxin, Pseudomonas exotoxin A, doxorubicin, methotrexate, an auristatin, a maytansine, a calicheamicin, a duocarmycin, a pyrrolobenzodiazepine dimer, and 7-ethyl-10-hydroxy-camptothecin.

21. The method of claim 18, wherein the antibody is linked to a modified immunoglobulin Fc domain modified to target the FcγRIIa receptor, the FcγRIIIa receptor, or the FcRn receptor.

22. The method of claim 18, wherein the antibody is linked to anti-CD3 or anti-CD16.
